SEQUENCE LISTING (6 sequences)

SEQ ID NO: 1 - DNA, 1344 bp, Homo sapiens
  CDS (1)..(1341): ADRA2B variant type sequence
atggaccaccaggacccctactccgtgcaggccacagcggccatagcggcggccatcaccttcctcattctctttaccatcttcggcaacgctctggtcatcctggctgtgttgaccagccgctcgctgcgcgcccctcagaacctgttcctggtgtcgctggccgccgccgacatcctggtggccacgctcatcatccctttctcgctggccaacgagctgctgggctactggtacttccggcgcacgtggtgcgaggtgtacctggcgctcgacgtgctcttctgcacctcgtccatcgtgcacctgtgcgccatcagcctggaccgctactgggccgtgagccgcgcgctggagtacaactccaagcgcaccccgcgccgcatcaagtgcatcatcctcactgtgtggctcatcgccgccgtcatctcgctgccgcccctcatctacaagggcgaccagggcccccagccgcgcgggcgcccccagtgcaagctcaaccaggaggcctggtacatcctggcctccagcatcggatctttctttgctccttgcctcatcatgatccttgtctacctgcgcatctacctgatcgccaaacgcagcaaccgcagaggtcccagggccaagggggggcctgggcagggtgagtccaagcagccccgacccgaccatggtggggctttggcctcagccaaactgccagccctggcctctgtggcttctgccagagaggtcaacggacactcgaagtccactggggagaaggaggagggggagacccctgaagatactgggacccgggccttgccacccagttgggctgcccttcccaactcaggccagggccagaaggagggtgtttgtggggcatctccagaggatgaagctgaagaggaggaagaggaggaggaggagtgtgaaccccaggcagtgccagtgtctccggcctcagcttgcagccccccgctgcagcagccacagggctcccgggtgctggccaccctacgtggccaggtgctcctgggcaggggcgtgggtgctataggtgggcagtggtggcgtcgaagggcgcacgtgacccgggagaagcgcttcaccttcgtgctggctgtggtcattggcgtttttgtgctctgctggttccccttcttcttcagctacagcctgggcgccatctgcccgaagcactgcaaggtgccccatggcctcttccagttcttcttctggatcggctactgcaacagctcactgaaccctgttatctacaccatcttcaaccaggacttccgccgtgccttccggaggatcctgtgccgcccgtggacccagacggcctggtga

SEQ ID NO: 2 - protein, 447 aa, Homo sapiens
MDHQDPYSVQATAAIAAAITFLILFTIFGNALVILAVLTSRSLRAPQNLFLVSLAAADILVATLIIPFSLANELLGYWYFRRTWCEVYLALDVLFCTSSIVHLCAISLDRYWAVSRALEYNSKRTPRRIKCIILTVWLIAAVISLPPLIYKGDQGPQPRGRPQCKLNQEAWYILASSIGSFFAPCLIMILVYLRIYLIAKRSNRRGPRAKGGPGQGESKQPRPDHGGALASAKLPALASVASAREVNGHSKSTGEKEEGETPEDTGTRALPPSWAALPNSGQGQKEGVCGASPEDEAEEEEEEEEECEPQAVPVSPASACSPPLQQPQGSRVLATLRGQVLLGRGVGAIGGQWWRRRAHVTREKRFTFVLAVVIGVFVLCWFPFFFSYSLGAICPKHCKVPHGLFQFFFWIGYCNSSLNPVIYTIFNQDFRRAFRRILCRPWTQTAW

SEQ ID NO: 3 - DNA, 1353 bp, Homo sapiens
  CDS (1)..(1350): ADRA2B wild type sequence
atggaccaccaggacccctactccgtgcaggccacagcggccatagcggcggccatcaccttcctcattctctttaccatcttcggcaacgctctggtcatcctggctgtgttgaccagccgctcgctgcgcgcccctcagaacctgttcctggtgtcgctggccgccgccgacatcctggtggccacgctcatcatccctttctcgctggccaacgagctgctgggctactggtacttccggcgcacgtggtgcgaggtgtacctggcgctcgacgtgctcttctgcacctcgtccatcgtgcacctgtgcgccatcagcctggaccgctactgggccgtgagccgcgcgctggagtacaactccaagcgcaccccgcgccgcatcaagtgcatcatcctcactgtgtggctcatcgccgccgtcatctcgctgccgcccctcatctacaagggcgaccagggcccccagccgcgcgggcgcccccagtgcaagctcaaccaggaggcctggtacatcctggcctccagcatcggatctttctttgctccttgcctcatcatgatccttgtctacctgcgcatctacctgatcgccaaacgcagcaaccgcagaggtcccagggccaagggggggcctgggcagggtgagtccaagcagccccgacccgaccatggtggggctttggcctcagccaaactgccagccctggcctctgtggcttctgccagagaggtcaacggacactcgaagtccactggggagaaggaggagggggagacccctgaagatactgggacccgggccttgccacccagttgggctgcccttcccaactcaggccagggccagaaggagggtgtttgtggggcatctccagaggatgaagctgaagaggaggaagaggaggaggaggaggaggaagagtgtgaaccccaggcagtgccagtgtctccggcctcagcttgcagccccccgctgcagcagccacagggctcccgggtgctggccaccctacgtggccaggtgctcctgggcaggggcgtgggtgctataggtgggcagtggtggcgtcgaagggcgcacgtgacccgggagaagcgcttcaccttcgtgctggctgtggtcattggcgtttttgtgctctgctggttccccttcttcttcagctacagcctgggcgccatctgcccgaagcactgcaaggtgccccatggcctcttccagttcttcttctggatcggctactgcaacagctcactgaaccctgttatctacaccatcttcaaccaggacttccgccgtgccttccggaggatcctgtgccgcccgtggacccagacggcctggtga

SEQ ID NO: 4 - protein, 450 aa, Homo sapiens
MDHQDPYSVQATAAIAAAITFLILFTIFGNALVILAVLTSRSLRAPQNLFLVSLAAADILVATLIIPFSLANELLGYWYFRRTWCEVYLALDVLFCTSSIVHLCAISLDRYWAVSRALEYNSKRTPRRIKCIILTVWLIAAVISLPPLIYKGDQGPQPRGRPQCKLNQEAWYILASSIGSFFAPCLIMILVYLRIYLIAKRSNRRGPRAKGGPGQGESKQPRPDHGGALASAKLPALASVASAREVNGHSKSTGEKEEGETPEDTGTRALPPSWAALPNSGQGQKEGVCGASPEDEAEEEEEEEEEEEECEPQAVPVSPASACSPPLQQPQGSRVLATLRGQVLLGRGVGAIGGQWWRRRAHVTREKRFTFVLAVVIGVFVLCWFPFFFSYSLGAICPKHCKVPHGLFQFFFWIGYCNSSLNPVIYTIFNQDFRRAFRRILCRPWTQTAW

SEQ ID NO: 5 - DNA, 20 bp, Artificial Sequence
  ADRA2B pcr primer f
gggtgtttgtggggcatctc

SEQ ID NO: 6 - DNA, 19 bp, Artificial Sequence
  ADRA2B pcr primer r
tggcactgcctggggttca

(19) United States
(12) Patent Application Publication
(10) Pub. No.: US 2006/0154249 A1
(43) Pub. Date: Jul. 13, 2006

(54) Method and kit for detecting a risk for diabetes or a metabolic syndrome

(75) Inventors: Jukka Salonen, Kuopio (FI); Mia Pirskanen, Kuopio (FI); Tomi-Pekka Tuomainen, Kuopio (FI); Faisel Yunus, Lahore (PK)

Correspondence Address: BIRCH STEWART KOLASCH & BIRCH, PO BOX 747, FALLS CHURCH, VA, 22040-0747, US

(73) Assignee: Oy Jurilab Ltd, Kuopio (FI)

(21) Appl. No.: 10/538,198

(22) PCT Filed: Dec. 11, 2003
(86) PCT No.: PCT/FI03/00946

§ 371 Date: Jan. 6, 2006

(30) Foreign Application Priority Data
Dec. 11, 2002 (FI) 20022178

(51) Int. Cl. C12Q 1/68

(52) U.S. Cl. 435/6

(57) ABSTRACT

The invention provides a method and kit for detecting or diagnosing a risk of or predisposition to diabetes or a metabolic syndrome in a subject, the method comprising the steps of providing a biological sample of the subject to be tested and detecting the presence or absence of a variant genotype of the human α_{2B}. adrenoceptor in the biological sample. The presence of the variant genotype indicates an increased risk of diabetes or a metabolic syndrome in said subject. The invention also relates to a method for the treatment of type 2 diabetes.

### FIELD OF THE INVENTION

This invention relates to a method for detecting or diagnosing a risk of, or predisposition to, hypertension in a subject, for targeting antihypertensive treatment in a subject and for selecting subjects for studies testing antihypertensive agents.

### BACKGROUND OF THE INVENTION

The publications and other materials used herein to illuminate the background of the invention, and in particular, to provide additional details with respect to the practice, are incorporated by reference.

The α₂-adrenoceptors (α₂-ARs) mediate many of the physiological effects of the catecholamines norepinephrine and epinephrine. Three genetic subtypes of α₂-adrenoceptors are known in humans and other mammals, denoted as α_{2A}-, α_{2B}- and α_{2C}-adrenoceptors. The human genes encoding the receptors are located on chromosomes 10, 2 and 4, respectively. No splice variants are known to exist of these receptors, as the genes are intronless. The tissue distributions and physiological and pharmacological functions of the receptor subtypes have been reviewed e.g. by MacDonald et al. (1997) and Docherty (1998). Based on recent studies with gene-targeted and transgenic mice, α_{2A}-adrenoceptors mediate most of the pharmacological actions ascribed to currently available α₂-adrenoceptor agonists, including inhibition of neurotransmitter release, central hypotensive and bradycardic effects, sedation and anesthesia, and analgesia. The same studies indicate that α_{2B}-adrenoceptors mediate peripheral pressor responses in response to agonist activation (Link et al. 1996, Macmillan et al. 1996) and thus play a significant role in the onset of hypertension (Calzada and Artinano 2001). Other physiological or pharmacological effects have not been associated with certainty with this receptor subtype. The α_{2C}-adrenoceptor subtype appears to be involved in regulation of complex behaviors. It is not known that this subtype would have important functions in peripheral tissues outside the central nervous system or in cardiovascular regulation.

Hypertension, like many other common disorders, arises from complex interactions between genetic and environmental factors. It is reasonable to assume that functionally important genetic variation in mechanisms important for the regulation of vascular functions will be found to be associated with the pathogenesis and therapy of hypertension. A variant form of the human α_{2B}-AR gene was recently identified (Heinonen et al., 1999). The variant allele encodes a receptor protein with a deletion of three glutamate residues in an acidic stretch of 18 amino acids (of which 15 are glutamates) located in the third intracellular loop of the receptor polypeptide. This acidic stretch is a unique feature in the primary structure of α_{2B}-AR in comparison to α_{2A}-AR and α_{2C}-AR, suggesting that the motif has a distinct role in the function of α₂B-AR. Amino acid sequence alignment of α_{2B}-AR polypeptides of different mammals reveals that the acidic stretch is highly conserved among the am-ARs of mammals and that the acidic stretch is long in humans in comparison to other species. This suggests that the motif is important for the functionality of the receptor, and that the short form (D for “deletion”) probably represents the ancestral form and the long form (I for “insertion”) could well represent a more recent allelic variant in humans. Jewell-Motz and Liggett (1995) studied the in vitro functions of this stretch using site-directed mutagenesis to delete as well as to substitute 16 amino acids of the stretch. Their results suggest that this acidic motif is necessary for full agonist-promoted receptor phosphorylation and desensitisation.

Based on the vasoconstrictive property of α_{2B}-AR in mice and the involvement of this acidic region in the desensitisation mechanism of the receptor, we hypothesised that the deletion variant confers reduced receptor desensitisation and therefore augmented vasoconstriction of systemic arteries that could be associated with hypertension. To test this hypothesis, we carried out a population study in 912 middle-aged Finnish men.

### SUMMARY OF THE INVENTION

The object of this invention is to provide a method for screening a subject to assess if an individual is at risk to develop hypertension, based on the genotype of α₂B-adrenoceptor gene and a method to target blood pressure lowering treatments. A further object of the invention is to provide a method for the selection of human subjects for studies testing antihypertensive effects of drugs.

The present invention concerns a method for detecting a risk of hypertension in a subject by determining the pattern of alleles encoding a variant α_{2B}-adrenoceptor, i.e. to determine if said subject's genotype of the human α_{2B}-adrenoceptor is of the deletion/deletion (DAD) type, comprising the steps of
a) providing a biological sample of the subject to be tested,
b) providing an assay for detecting in the biological sample the presence of
   i) the insertion/insertion (I/I) or deletion/insertion (D/I) genotypes of the human α_{2B}-adrenoceptor, or
   ii) the D/D genotype of the human α_{2B}-adrenoceptor, the presence of the D/D genotype indicating an increased risk of hypertension in said subject.

According to the invention, the method allows for establishing whether the said subject is of said D/D genotype or not, a presence in the biological sample, such as a blood sample or a buccal sweep, of said D/D genotype thus indicating an increased risk of the subject to develop hypertension, and/or indicating the subject being in need for treatment, such as α_{2B}-selective or α_{2B}-nonselective α₂-adrenoceptor antagonist therapy.

The said method can thus include a step of identifying a subject having an increased risk to develop hypertension, and/or a subject in need of therapy, such as α_{2B}-selective or α_{2B}-nonselective α₂-adrenoceptor antagonist therapy for hypertension.

The invention also concerns a method as defined comprising the further steps of
c) assessing at least one of the two following
   i) the subject's risk to develop hypertension, or
   ii) the subject's need for α_{2B}-selective or α_{2B}-nonselective α₂-adrenoceptor antagonist therapy for hypertension,
based on whether said subject is of said D/D genotype or not.

A further object of the invention is a method for treating, or targeting the treatment of hypertension in a hypertensive subject by determining the pattern of alleles encoding a variant α_{2B}-adrenoceptor, i.e. by determining if said subject's genotype of the human α_{2B}-adrenoceptor is of the deletion/deletion (D/D) type, comprising the steps presented above, and treating a subject of the D/D genotype with a drug affecting the noradrenaline sensitivity or sympathetic activity of the subject.

The present invention is also directed to a kit for detecting a risk of hypertension in a subject, or for selecting a subject for targeting antihypertensive treatment or studies for testing antihypertensive agents, comprising means for determining the pattern of alleles encoding a variant α_{2B}-adrenoceptor in a biological sample, as well as its use.

The invention also provides a DNA sequence comprising a nucleotide sequence encoding a variant α_{2B}-adrenoceptor protein with a deletion of at least 1 glutamate from a glutamic acid repeat element of 12 glutamates, amino acids 298-309, in an acidic stretch of 18 amino acids 294-311, located in the 3^{rd} intracellular loop of the receptor polypeptide.

The invention further provides a variant α_{2B}-adrenoceptor protein with a deletion of at least 1 glutamate from a glutamic acid repeat element of 12 glutamates, amino acids 298-309, in an acidic stretch of 18 amino acids 294-311, located in the 3^{rd} intracellular loop of the receptor polypeptide.

### DETAILED DESCRIPTION OF THE INVENTION

The present invention relates to a DNA molecule encoding a variant human α_{2B}-adrenoceptor, said variant α_{2B}-adrenoceptor protein and a method to assess the risk of individuals to develop hypertension in mammals as well as a method for the targeting treatment for hypertension.

The word treating shall also be understood to include preventing.

The concept “a deletion of at least 1 glutamate from a glutamic acid repeat element of 12 glutamates” refers to any deletion of 1 to 12 glutamates irrespective of the specific location in, or how many glutamates from said repeat element of 12 glutamates, amino acids 298-309 (SEQ ID NO: 4), in an acidic stretch of 18 amino acids 294-311 located in the 3^{rd} intracellular loop of the receptor polypeptide are deleted.

The concept “deletion/deletion (D/D) genotype of the human α_{2B}-adrenoceptor”, in short “D/D genotype”, refers to a genotype of an individual having both α_{2B}-adrenoceptor alleles code for a variant α_{2B}-adrenoceptor with a deletion of at least 1 glutamate from a glutamic acid repeat element of 12 glutamates, amino acids 298-309, in an acidic stretch of 18 amino acids 294-311 (SEQ ID NO: 4), located in the 3^{rd} intracellular loop of the receptor polypeptide. Correspondingly “deletion/insertion (D/I) genotype” refers to a genotype having one of the gene alleles code for an α_{2B}-adrenoceptor with a said deletion and the other without a said deletion, i.e. with a respective insertion, and thus the “insertion/insertion (I/I) genotype” refers to a genotype having both alleles code for an α_{2B}-adrenoceptor without said deletion or deletions.

A common variant form (SEQ ID NO: 1) of the human α_{2B}-AR gene (SEQ ID NO: 3) was recently identified (Heinonen et al. 1999). This variant gene encodes a receptor protein (SEQ ID NO: 2) with a deletion of 3 glutamates, amino acids 307-309, from a glutamic acid (Glu) repeat element of 12 glutamates, amino acids 298-309, in an acidic stretch of 18 amino acids 294-311 (SEQ ID NO: 4), located in the 3^{rd} intracellular loop of the receptor polypeptide. This variant gene (SEQ ID NO: 1) was associated with decreased basal metabolic rate (BMR) in a group of obese Finnish subjects (Heinonen et al. 1999). Of the 166 obese subjects, 47 (28%) were homozygous for the long 12 glutamate repeat element (Glu¹²/Glu¹²), whereas 90 (54%) were heterozygous (Glu¹²/Glu⁹) and 29 (17%) were homozygous for the short form (Glu⁹/Glu⁹).

The results to be presented below show that in a population sample of 912 Finnish middle-aged men subjects homozygous for the short form (Glu⁹/Glu⁹) described above, thus representing a deletion/deletion (D/D) genotype of the α_{2B}-adrenoceptor, have a significantly elevated risk for hypertension. Based on these results and previous publications referred to above it can be postulated that this DAD genotype is related to an impaired capacity to downregulate α_{2B}-adrenoceptor function during sustained receptor activation. Since altered α_{2B}-adrenoceptor function seems to be of relevance in the pathogenesis of hypertension, we believe it could also be of relevance in subjects with the insertion/deletion (I/D) (heterozygous Glu¹²/Glu⁹) and insertion/insertion (I/I) (homozygous Glu¹²/Glu¹²) genotypes when other risk factors for hypertension are present. Further, since this specific deletion of 3 glutamates from said glutamic acid repeat element of 12 glutamates, amino acids 298-309, in said acidic stretch of 18 amino acids 294-311, located in the 3^{rd} intracellular loop of the receptor polypeptide seems to be of relevance in hypertension we believe that also other deletions, i.e. deletions of at least 1 glutamate, from said glutamic acid repeat element of 12 glutamates, amino acids 298-309, could be of relevance in the pathogenesis of hypertension, because the 3^{rd} intracellular loop of the receptor polypeptide it is located in seems to have an essential role in the down-regulation of the α_{2B}-adrenoceptor. Thus persons with the DID genotype have chronically up-regulated α_{2B}-adrenoceptors, leading to the elevation of systemic blood pressure.

α_{2B}--adrenoceptors mediate contraction of arteries, and genetic polymorphism present in the α_{2B}-adrenoceptor gene renders some subjects more susceptible to α_{2B}-adrenoceptor mediated vasoconstriction of the blood pressure regulating resistance arteries (arteriolies) and associated clinical disorders such as hypertension. These subjects will especially benefit from treatment with an α_{2B}-adrenoceptor antagonist, and will be at increased risk for adverse effects if subtype-nonselective α₂-agonists are administered to them. Therefore, a gene test recognizing subjects with a deletion variant of the α_{2B}-adrenoceptor gene will be useful in diagnostics and patient selection for specific therapeutic procedures and clinical drug testing trials. A gene test recognizing the D/D genotype of the α_{2B}-adrenoceptor is useful in assessing an individual's risk to develop hypertension related to the D/D genotype. The test can be used to set a specific subdiagnosis of hypertension, based on its genetic etiology.

Furthermore, a gene test recognizing the D/D genotype of the α_{2B}-adrenoceptor is useful in selecting drug therapy for patients with hypertension. Such drugs are e.g. a drug modulating, inhibiting or activating the vascular alpha- or beta-adrenargic receptors of the subjects either directly or through central nervous system effects, for example pindolol, propranolol, sotalol, timolol, acebutolol, atenol, betaxolol, bisoprol, esmolol, metoprolol, seliprol, carvedilol, labetalol, clonidine, moxonidine, prazosin, or indapamid, including α-adrenoceptor antagonists (α_{2B}-selective or nonselective).

For instance, as angiotensin II causes an increase of noradrenaline sensitivity, and this effect is at least in part mediated by α-adrenoceptors (Datte et al. 2000), the blood pressure lowering effect of drugs acting through angiotensin II inhibition, such as the angiotensin (AT) receptor blockers, is conceivably enhanced in persons with the D/D genotype of the α_{2B}-adrenoceptor. Such drugs are for example captopril, cinapril, enalapril, imidapril, lisinopril, moexipril, perindopril, ramipril, trandolapril, candesartan, eprosartan, irbesartan, losartan, valsartan or telmisartan.

A gene test recognizing the D/D genotype of the α_{2B}-adrenoceptor is useful in selecting drug therapy for patients who might be at increased risk for adverse effects of α₂-adrenergic agonists; either it will be possible to avoid the use of α₂-agonists in such patients, or it will be possible to include a specific α_{2B}-antagonist in their therapeutic regimen.

On the other hand, it is conceivable that the patients with other than the D/D genotype will benefit more from other antihypertensive drugs.

The DNA sequence can be used for screening a subject to determine if said subject is a carrier of a variant gene. The determination can be carried out either as a DNA analysis according to well known methods, which include direct DNA sequencing of the normal and variant gene, allele specific amplification using the polymerase chain reaction (PCR) enabling detection of either normal or variant sequence, or by indirect detection of the normal or variant gene by various molecular biology methods including e.g. PCR-single stranded conformation polymorphism (SSCP) method or denaturing gradient gel electrophoresis (DGGE). Determination of the normal or variant gene can also be done by using a restriction fragment length polymorphism (RFLP) method, which is particularly suitable for genotyping large numbers of samples. Similarly, a test based on gene chip or array technology can be easily developed in analogy with many currently existing such tests for single-nucleotide polymorphisms.

The determination can also be carried out at the level of RNA by analyzing RNA expressed at tissue level using various methods. Allele specific probes can be designed for hybridization. Hybridization can be done e.g. using Northern blot, RNase protection assay or in situ hybridization methods. RNA derived from the normal or variant gene can also be analyzed by converting tissue RNA first to cDNA and thereafter amplifying cDNA by an allele specific PCR method.

The kit for use in the method according to the invention preferably contains the various components needed for carrying out the method packaged in separate containers and/or vials and including instructions for carrying out the method. Thus, for example, some or all of the various reagents and other ingredients needed for carrying out the determination, such as buffers, primers, enzymes, control samples or standards etc can be packaged separately but provided for use in the same box. Instructions for carrying out the method can be included inside the box, as a separate insert, or as a label on the box and/or on the separate vials. The kit may also contain the necessary software needed to interpret the results obtained with the kit, or for utilizing the results from a gene chip used in the method.

The invention will be described in more detail in the experimental section.

### Experimental Section

Determination of Genomic Alleles Encoding the a α_{2B}-Adrenoceptor

PCR-SSCA Analysis

The polymerase chain reaction-single stranded conformational analysis (PCR-SSCA) used to identify the genomic alleles encoding the α_{2B}--adrenoceptor was carried out as follows: The genomic DNA encoding the α_{2B}-adrenergic receptor was amplified in two parts specific for the intronless α_{2B}-adrenoceptor gene sequence (Lomasney et al. 1990). The PCR primer pairs for PCR amplification were as follows: Pair 1: 5′-GGGGCGACGCTCTTGTCTA-3′ (SEQ ID NO: 5) and 5′-GGTCTCCCCCTCCTCCTTC-3′ (SEQ ID NO: 6) (product size 878 bp), pair 2: 5′-GCAGCAACCGCAGAGGTC-3′ (SEQ ID NO: 7) and 5′-GGGCAAGAAGCAGGGTGAC-3′ (SEQ ID NO: 8) (product size 814 bp). The primers were delivered by KeboLab (Helsinki, Finland). PCR amplification was conducted in a 5 μl volume containing 100 ng genomic DNA (isolated from whole blood), 2.5 mmol/l of each primer, 1.0 mmol/l deoxy-NTPs, 30 nmol/l ³³P-dCTP and 0.25 U AmpliTaq DNA polymerase (Perkin Elmer Cetus, Norwalk, Conn.). PCR conditions were optimized using the PCR Optimizer™ kit (Invitrogen, San Diego, Calif.). Samples were amplified with a GeneAmp PCR System 9600 (Perkin Elmer Cetus). PCR products were digested with restriction enzymes for SSCA analysis. The product of primer pair 1 was digested with Dde I and Dra III (Promega Corp., Madison, Wis.). The product of primer pair 2 was digested with Alu I and Hinc II (Promega Corp.). The digested samples were mixed with SSCA buffer containing 95% formamide, 10 mmol/l NaOH, 0.05% xylene cyanol and 0.05% bromophenol blue (total volume 25 μl). Before loading, the samples were denatured for 5 min at 95° C. and kept 5 min on ice. Three microliters of each sample were loaded on MDE™ high-resolution gel (FMC, BioProducts, Rockland, Mass.). The gel electrophoresis was performed twice, at two different running conditions: 6% MDE gel at +4° C. and 3% MDE gel at room temperature, both at 4 W constant power for 16 h. The gels were dried and autoradiography was performed by apposing to Kodak BioMax MR film for 24 h at room temperature.

Sequencing and Genotyping

DNA samples migrating at different rates in SSCA were sequenced with the Thermo Sequenase™ Cycle Sequencing Kit (Amersham Life Science, Cleveland, Ohio).

For genotyping the identified 3-glutamic acid deletion, DNA was extracted from peripheral blood using standard methods. The α_{2B}-AR I/D genotype was determined by separating PCR-amplified DNA fragments with. electrophoresis. Based on the nature of the I/D variant, identification of the long and short alleles was achieved by their different electrophoretic migration rates due to their 9 bp size difference.

The region of interest was amplified using a sense primer 5′-AGG-GTG-TTT-GTG-GGG-CAT-CT-3′ (SEQ ID NO:9) and an anti-sense primer 5′-CAA-GCT-GAG-GCC-GGA-GAC-ACT-3′ (SEQ ID NO: 10)(Oligold, Eurogentec, Belgium), yielding a product size of 112 bp for the long allele (I) and 103 bp for the short allele (D). PCR amplification was conducted in a 10 μl volume containing ˜100 ng genomic DNA, 1× buffer G (Invitrogen, San Diego, Calif., USA), 0.8 mM dNTs, 0.3 μM of each primer and 0.25 units of AmpliTaq DNA polymerase (Perkin Elmer Cetus, Norwalk, Conn., USA). Samples were amplified with a GeneAmp PCR System 9600 (Perkin Elmer Cetus). After initial denaturation at 94° C. for 2 minutes, the samples were amplified over 35 cycles. PCR amplification conditions were 96° C. (40 s), 69° C. (30 s) and 72° C. (30 s) followed by final extension at 72° C. for 6 minutes. The PCR products representing the long and short alleles were identified by two alternative methods. 1) The amplified samples were mixed with 4 μl of stop solution (Thermo Sequenase™ Cycle Sequencing kit), heated to 95° C. for 2 min, and loaded hot onto sequencing gels (Long Ranger™, FMC). The gels were dried and autoradiography was performed as previously described. 2) Separation of the amplified PCR products was performed with electrophoresis on a high-resolution 4% Metaphor agarose gel (FMC Bioproducts, Rockland, Me.) and the bands were visualized by ethidium bromide staining. In both methods, the long (Glu¹²) and short (Glu⁹) alleles were identified based on their different electrophoretic migration rates.

Population Study

The above referred population study of 912 Finnish middle-aged men subjects including 192 subjects with a specific deletion/deletion (D)/D) genotype of the α_{2B}-adrenoceptor is described in more detail in the following:

Knowing the vasoconstrictive property of α_{2B}-AR in mice and the possible involvement of the investigated acidic region in the desensitization mechanism of the receptor we hypothesized that the observed insertion/deletion allelic variation could be associated with hypertension. To test this hypothesis, we carried out a population study in 912 middle-aged Finnish men with no prior history of coronary heart disease. The study was carried out as part of the Kuopio Ischemic Heart Disease Risk Factor Study (KMID), which is an ongoing population-based study designed to investigate risk factors for cardiovascular diseases and related outcomes in men from eastern Finland (Salonen 1988). This area is known for its homogenous population (Sajantila et al. 1996) and high coronary morbidity and mortality rates (Keys 1980).

Of the 912 subjects, 192 (21%) had the D/D genotype, 256 (28%) had the I/I genotype and 464 (51%) were heterozygous i.e. I/D. This genotype distribution is in Hardy-Weinberg equilibrium (p=0.46).

Four hundred and seventeen men had no family history of hypertension, and 495 had hypertension in the family (either parents or siblings or both). It was assumed that genetic traits would have a stronger association with hypertension in the subjects who had a history of hypertension in the family, and thus the association between the α_{2B}-adrenoceptor genotype and hypertension was analyzed separately in men with and without family history (Tables 1 and 2). In a multivariate linear regression model, men with the DD genotype had on the average a higher mean systolic blood pressure (BP) as compared with the other genotypes (p=0.021) among men with a family history (Table 1). Among those with a family history of hypertension, DD homozygous men had, in a multivariate logistic model, a 2.04-fold (95% confidence interval 1.06 to 3.93, p=0.032) probability (prevalence) of hypertension (either systolic BP at least 165 mmHg or diastolic BP at least 95 mmHg or antihypertensive medication, Table 2).

The association of the use of α-adrenoceptor antagonists such as prazosin with hypertension was analyzed among the DD homozygous men and other men, separately. The antihypertensive effect was estimated as the blood pressure difference between the specific drug type vs other drugs. In men with the DD genotype but not among the other men, the use of α-adrenoceptor antagonists was associated with a lowering of both systolic and diastolic blood pressure as well as decreased occurrence of a number of self-reported adverse effects. Among 440 men who were hypertensive at the 11-year follow-up (systolic BP≧165 mmHg or diastolic BP≧95 mmHg or antihypertensive treatment), among men with the D/D α_{2B}-adrenoceptor genotype, the means systolic BP was 111 mmHg in those treated with alpha-blocker and 137 mmHg in those treated with other drugs, whereas these means were 150 mmHg and 138 mmHg in men with other genotypes. There was a similar trend for beta-adrenoceptor antagonists (beta-blockers) such as atenolol, metoprolol and pindolol, as well as for angiotensin converting enzyme (ACE) inhibitors such as captopril, enalapril and lisinopril. For example, among 344 men, who were hypertensive in the KIHD baseline examination the mean systolic blood pressure was 11 years later among subjects with DAD genotype 134 mmHg in those treated with beta-blocker and 141 mmHg among those treated with other drugs, whereas for men with other genotypes these means were 137 and 138 mmHg. Among the 440 men who were hypertensive at the 11-year follow-up, in those with the D/D genotype, the mean systolic BP was 133 in beta-blocker treated and 139 in others, whereas in men with other genotypes these means were 139 and 138 mmHg. Among men who were treated with β-blockers, the mean systolic blood pressure was 128.8 (SD 16.2) in those with the D/D genotype and 135.5 mmHg (SD 19.3) in those with other genotypes (p=0.04 for difference). In a linear covariance model adjusting for age and body-mass index (kg/m2), the genotype-β-blocker interaction was statistically significant (1-sided p=0.04).

The antihypertensive effect of antihypertensive drug types acting through other mechanisms than adrenoceptor or noradrenaline sensitivity modulation and was greater in men with other than the DID genotype. For example, the blood pressure lowering effect of diuretics and calcium channel blockers was larger in α_{2B}-AR genotypes other than D/D. Men with the DD genotype had an increased prevalence of adverse effects and a smaller antihypertensive response during α₂-adrenoceptor agonist therapy such as clonidin.

Taken together, the known biological properties of the α_{2B}-AR, the homogeneity of the Finnish population, the study design, the relatively large representative study population and the association of hypertension with one trait suggest that the D/D receptor allele is a causal genetic risk factor for hypertension.

It will be appreciated that the methods of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

TABLE 1
The strongest risk factors for the mean systolic blood pressure in linear regression models
among men with no family history of hypertension and in those with a family history.

| | No family history of hypertension | | | Family history of hypertension | | |
|---|---|---|---|---|---|---|
| Risk factor | Coefficient | 95% CI | P | Coefficient | 95% CI | P |
| α_{2B}-AR genotype (DD vs. other) | −0.44 | −3.83, 2.95 | 0.799 | 3.87 | 0.59, 7.16 | 0.021 |
| Body-mass index (kg/m2) | 1.06 | 0.67, 1.46 | <0.001 | 0.84 | 0.44, 1.24 | <0.001 |
| Age (years) | 0.62 | 0.40, 0.85 | <0.001 | 0.77 | 0.57, 0.98 | <0.001 |
| Resting heart rate (bpm) | 0.19 | 0.05, 0.32 | 0.006 | 0.36 | 0.22, 0.49 | <0.001 |
| Fasting blood glucose (umol/L) | 0.85 | −0.37, 2.07 | 0.172 | 2.23 | 0.89, 3.56 | 0.001 |
| Alcohol from beer (g/wk) | 0.002 | −0.002, 0.03 | 0.092 | 0.04 | 0.02, 0.06 | 0.001 |
| Use of beta-blocking agent (yes vs. no) | −3.0 | −9.0, 3.0 | 0.327 | 6.9 | 2.5, 11.2 | 0.002 |
| Family history of cancer | −1.6 | −4.8, 1.5 | 0.306 | 2.0 | −1.1, 5.1 | 0.200 |
| R square for the model | 0.170 | | | 0.261 | | |

TABLE 2
Probability of systolic hypertension and its 95% confidence interval,
related with α□_{2B}-AR genotype and other strongest risk
factors in men free of family history of hypertension and in those with a
family history. Results are from logistic regression models.

| | No family history of hypertension | | | Family history of hypertension | | |
|---|---|---|---|---|---|---|
| | Relative | | | Relative | | |
| Risk factor | risk | 95% CI | P | risk | 95% CI | P |
| α_{2B}-AR genotype (DD vs. other) | 0.61 | 0.22, 1.67 | 0.333 | 2.04 | 1.06, 3.93 | 0.032 |
| Body-mass index (kg/m2) | 1.23 | 1.12, 1.36 | <0.001 | 1.11 | 1.02, 1.21 | 0.015 |
| Age (years) | 1.12 | 1.04, 1.20 | 0.001 | 1.10 | 1.05, 1.15 | <0.001 |
| Resting heart rate (bpm) | 0.98 | 0.95, 1.02 | 0.384 | 1.03 | 1.003, 1.06 | 0.032 |
| Fasting blood glucose (umol/L) | 1.25 | 0.97, 1.60 | 0.085 | 1.28 | 1.03, 1.60 | 0.027 |
| Alcohol from beer (g/wk) | 1.00 | 1.00, 1.01 | 0.083 | 1.004 | 1.00, 1.01 | 0.036 |
| Use of beta-blocking agent (yes vs. no) | 0.001 | 0.00, 521 | 0.725 | 3.09 | 1.42, 6.70 | 0.004 |
| Family history of cancer | 0.57 | 0.23, 1.45 | 0.242 | 2.02 | 1.07, 3.82 | 0.031 |
| Number of hypertensives | 31 | | | 60 | | |
| Number of men | 417 | | | 495 | | |
| R square for the model | 0.202 | | | 0.219 | | |

### REFERENCES

Calzada B C, Artinano A L. Alpha-adrenoceptor subtypes. Pharmacol Res 2001;44:195-208.

Datte J-Y, Gohlke P, Pees C, Ziegler A. Short treatments of normotensive and hypertensive rats by angiotensin II and nitric oxide inhibitor induce and increase of noradrenaline sensitivity in isolated vena portae preparations. Pharmacol Res 2000;41:641-648.

Docherty J R: Subtypes of functional α₁- and α₂-receptors. Eur J Pharmacol 1998;361:1-15

Heinonen P, Koulu M, Pesonen U, Karvonen M, Rissanen A, Laakso M, Valve R, Uusitupa M, Scheinin M: Identification of a three amino acid deletion in the alpha-2B-adrenergic receptor which is associated with reduced basal metabolic rate in obese subjects. J Clin Endocrinol Metab 1999;84:2429-2433

Jewell-Motz E, Liggett S B: An acidic motif within the third intracellular loop of the alpha2C2 adrenergic receptor is required for agonist-promoted phosphorylation and desensitization. Biochemistry 1995;34:11946-11953

Keys A: Seven Countries: A Multivariate Analysis of Death and Coronary Heart Disease. Cambridge, Mass., Harvard University Press, 1980

Link R E, Desai K, Hein L, Stevens M E, Chruscinski A, Bernstein D, Barsh G S, Kobilka B K: Cardiovascular regulation in mice lacking alpha2-adrenergic receptor subtypes b and c. Science 1996;273:803-805.

Lomasney J W, Lorenz W, Allen L F, King K, Regan J W, Yang-Feng T L, Caron M C, Lefkowitz R J: Expansion of the alpha-2 adrenergic receptor family: cloning and characterization of a human alpha-2 adrenergic receptor subtype, the gene for which is located on chromosome 2. Proc Natl Acad Sci USA. 1990;87:5094-5098.

MacDonald E, Kobilka B K, Scheinin M: Gene targeting—homing in on α₂-adrenoceptor subtype function. Trends Pharmacol Sci 1997;18:211-219

MacMillan L B, Hein L, Smith M S, Piascik M T, Limbird L E: Central hypotensive effects of the alpha2a-adrenergic receptor subtype. Science 1996;273:801-803

Sajantila A, Salem A H, Savolainen P, Bauer K, Gierig C, Paabo S: Paternal and maternal DNA lineages reveal a bottleneck in the founding of the Finnish population. Proc. Natl. Acad. Sci. U.S.A. 1996;93:12035-12039

Salonen J T: Is there a continuing need for longitudinal epidemiologic research? The Kuopio Ischaemic Heart Disease Risk Factor Study. Ann. Clin Res 1988;20:46-50

1. A method for detecting a risk of hypertension in a subject by determining the pattern of alleles encoding a variant 2B-adrenoceptor, comprising the steps of
a) providing a biological sample of the subject to be tested,
b) providing an assay for detecting in the biological sample the presence of
i) the insertion/insertion (I/I) or deletion/insertion (D/I) genotypes of the human α_{2B}-adrenoceptor, or
ii) the D/D genotype of the human α_{2B}-adrenoceptor, the presence of the D/D genotype indicating an increased risk of hypertension in said subject.

2. The method according to claim 1, wherein the assay is a DNA-assay.

3. The method according to claim 1 or 2, wherein the assay is carried out using a gene or DNA chip, microarray, strip, panel or similar combination of more than one genes, mutations or RNA expressions to be assayed.

4. The method according to claim 1, wherein the allelic pattern is determined using polymerase chain reaction.

5. The method according to claim 1, wherein the biological sample is a blood sample or buccal sweep sample and genomic DNA is isolated from the said sample.

6. The method according to claim 1, wherein the assay is based on a capturing probe which comprises a single strand of the cDNA, comprising a nucleotide sequence encoding a variant α_{2B}-adrenoceptor protein with a deletion of at least 1 glutamate from a glutamic acid repeat element of 12 glutamates, amino acids 298-309, in an acidic stretch of 18 amino acids 294-311, located in the 3^{rd} intracellular loop of the receptor polypeptide.

7. The method according to claim 1, wherein the assay is based on a capturing probe which comprises a single strand of the cDNA corresponding to the α_{2B}-adrenoceptor without the deletion of a glutamate from a glutamic acid repeat element of 12 glutamates, amino acids 298-309, in an acidic stretch of 18 amino acids 294-311, located in the 3^{rd} intracellular loop of the receptor polypeptide.

8. The method according to claim 1, wherein the said method is used for determining whether a subject will benefit from treatment with a drug affecting the noradrenaline sensitivity or sympathetic activity of the subject.

9. The method according to claim 1, wherein the said method is used for determining whether a subject will benefit from treatment with an α_{2B}-adrenoceptor antagonist.

10. The method according to claim 1, wherein the said method is used for determining whether a subject will be at increased risk of adverse effects if subtype-nonselective α₂-agonists or a diuretic or a calcium channel blocker are administered to them.

11. The method according to claim 1, comprising the step of selecting a subject of the D/D genotype for clinical drug trials testing the antihypertensive effects of compounds.

12. The method according to claim 11, wherein the said compound is a drug affecting the noradrenaline sensitivity or sympathetic activity of the subject.

13. The method according to claim 8 or 11, wherein the said compound is a drug modulating, inhibiting or activating the vascular alpha- or beta-adrenergic receptors of the subjects either directly or through central nervous system effects.

14. The method according to claim 8 or 11, wherein the said compound is an angiotensin converting enzyme (ACE) inhibitor, angiotensin II inhibitor or angiotensin receptor inhibitor.

15. The method according to claim 13, wherein the said compound is an α_{2B}-selective or α_{2B}-nonselective α₂-adrenoceptor antagonist.

16. A method for targeting the treatment of hypertension in a hypertensive subject by determining the pattern of alleles encoding a said variant α_{2B}-adrenoceptor, i.e. by determining if said subject's genotype of the human α_{2B}-adrenoceptor is of the deletion/deletion (D/D) type, comprising the steps presented in claim 1, and treating a subject of the D/D genotype with a drug affecting the noradrenaline sensitivity or sympathetic activity of the subject.

17. The method according to claim 16, wherein the said drug is a drug modulating, inhibiting or activating the vascular alpha- or beta-adrenergic receptors of the subjects either directly or through central nervous system effects.

18. The method according to claim 17, wherein the said drug is pindolol, propranolol, sotalol, timolol, acebutolol, atenol, betaxolol, bisoprol, esmolol, metoprolol, seliprol, carvedilol, labetalol, clonidine, moxonidine, prazosin, or indapamid.

19. The method according to claim 16, wherein the said drug is an angiotensin converting enzyme (ACE) inhibitor, angiotensin II inhibitors or angiotensin receptor inhibitor.

20. The method according to claim 19, wherein the said drug is captopril, cinapril, enalapril, imidapril, lisinopril, moexipril, perindopril, ramipril, trandolapril, candesartan, eprosartan, irbesartan, losartan, valsartan or telmisartan.

21. A method according to claim 17, wherein the said drug is an Cc2B-selective or α_{2B}-nonselective α₂-adrenoceptor or α-adrenoceptor antagonist.

22. A kit for detecting a risk of hypertension in a subject, or for selecting a subject for targeting antihypertensive treatment, or for selecting a subject for clinical drug trials testing the antihypertensive effect of compounds, comprising means for determining the pattern of alleles encoding a variant α_{2B}-adrenoceptor in a biological sample from said subject, and optionally software to interpret the results of the determination.

23. The use of the kit according to claim 22 for detecting a risk of hypertension in a subject,or for selecting a subject for targeting antihypertensive treatment, or for selecting a subject for clinical drug trials testing the antihypertensive effect of compounds.
